(19) United States
(12) Patent Application Publication
(10) Pub. No.: US 2003/0077600 A1
(43) Pub. Date: Apr. 24, 2003

(54) Photovoltaic device to accelerate the interaction among biomolecules

(76) Inventors: Chi-Chan Chen, Taipei (TW); Sung-Kay Chiu, Taipei (TW); Chi-Meng Tzeng, Taipei (TW); Wei-Chi Ku, Taipei (TW)

Correspondence Address: Kent H. Cheng, Esq., Cohen, Pontani, Lieberman & Pavane, Suite 1210, 551 Fifth Avenue, New York, NY, 10176, US

(21) Appl. No.: 10/045,306

(22) Filed: Oct. 23, 2001

(51) Int. Cl. C12Q 1/68; G01N 33/53; G01N 33/542; C12M 1/34

(52) U.S. Cl. 435/6; 435/7.9; 435/287.2; 204/461

(57) ABSTRACT

A photovoltaic device is employed to facilitate interactions between immobilized and dissolved biological materials. Such interactions including nucleotide hybridization, peptide-peptide interaction and peptide/nucleotide binding. Through the force of the photovoltaic effect, which is caused by illuminating the photovoltaic device with a light source, the efficiency of interaction between immobilized biomedical materials on the surface of the device and free biological materials is remarkably enhanced. Such photovoltaic device may be a microfabricated device having an array of microlocations capable of being used as a biological screening tool with high throughput applications.

### BACKGROUND OF THE INVENTION

1. Field of the Invention

This invention is related to devices and systems that facilitate active biological operations. More particularly, these biological operations include various nucleic acid hybridization and associated biopolymer interactions. Additionally, antibody/antigen reactions and other clinical diagnostics can be performed.

2. Description of the Related Art

Molecular biology comprises a wide variety of techniques for clinical diagnostic assays, such as nucleic acid hybridization analysis, restriction enzyme analysis, genetic sequence analysis and so on. One of the crucial techniques for these assays is to effectively and efficiently conduct multi-step, multiplex molecular biological reactions. Especially when these micro-scale biological reactions are highly selective and are conducted with relatively low concentration or quality of target molecules, there is a need to concentrate and drive the pertinent reactants to a specific location to enable or enhance these reactions. During or after the desired reaction, nonspecific analytes or molecules present at the specific location of the reaction need to be unreacted and/or eliminated.

Many attempts have been made to facilitate or control biological reactions. For example, attempts include those disclosed in Pace, U.S. Pat. No. 4,908,112, and Soane, U.S. Pat. No. 5,126,022. Heller et al., U.S. Pat. No. 5,605,662; and U.S. Pat. No. 5,849,486, describe a microelectronic device designed and fabricated to actively carry out and control multi-step and multiplex molecular biological reactions in microscopic formats. These reactions include nucleic acid hybridization, antibody/antigen reaction, diagnostics, and biopolymer synthesis. The device can electronically control the transport and attachment of specific binding entities to specific micro-locations. The specific binding entities include molecular biological molecules such as nucleic acids and polypeptides. The device can subsequently control the transport and reaction of analytes or reactants at the specific microlocations. The device is able to concentrate analytes and reactants, remove non-specifically bound molecules, provide stringency control for DNA hybridization reactions, and improve the detection of analytes.

As disclosed in U.S. Pat. No. 5,605,662, each of the microlocations needs to be connected with electric wires to provide the desired electrical potential. However, the interconnection of the wires on the chip limits the number of the microlocations. Additionally, the complex structure of the device increases the cost.

### SUMMARY OF THE INVENTION

The present invention relates to the design, fabrication, and use of a photovoltaic device and a system that can actively carry out biological operations. These operations include, but are not limited to, most molecular biological procedures, such as nucleic acid hybridization, antibody/antigen reaction, and related clinical diagnostics. The claimed devices and systems can carry out multi-step combinatorial biopolymer procedures, including, but not limited to, the interaction among different oligonucleotides, cells or peptides at specific microlocations on a given device.

The devices and systems of the present invention are preferably fabricated using photolithography techniques. The photovoltaic device of the present invention comprises a semiconductor substrate, which is coated with metal thin films. The metal thin films can be patterned by photolithography techniques to form an array of microlocations. A dielectric thin film can be deposited on the photovoltaic device and patterned by photolithography techniques to isolate the microlocations.

The device of the present invention is able to control and actively carry out a variety of assays and reactions. Charged biological species can be transported onto the microlocations by the induced electric field resulting from illuminating the microlocations. The charged biological species can be concentrated and reacted with the specific binding species at the microlocation. In the case of hybridization analysis, the sensitivity for detecting a specific analyte or reactant is tremendously improved because hybridization reactants are concentrated at a specific microscopic location. Any unbound analytes or reactants can be removed by applying a voltage on a device. Thus, the device also improves the specificity of the reactions.

The various features of novelty, which characterize the invention, are pointed out with particularity in the claims annexed to and forming a part of the disclosure. For a better understanding of the invention, its operating advantages, and specific objects attained by its use, reference should be had to the drawing and descriptive matter in which there are illustrated and described preferred embodiments of the invention.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings:

FIG. 1 shows the photovoltaic device of the present invention in cross-section view.

FIG. 2 shows a cross-section of the photovoltaic device of the present invention placed upside down from that displayed in FIG. 1, and on top of a transparent plate of sapphire that is coated with a thin film of indium tin oxide and a side border of gold.

FIG. 3 shows a top view of the microlocations overtime as nucleotide hybridization proceeded at the microlocations while the microlocations were illuminated by a laser source.

### DETAILED DESCRIPTION OF THE PRESENTLY PREFERRED EMBODIMENTS

1. Overview of the Invention

The photovoltaic devices of the present invention utilize the photo electromotive force of the p-n junction, heterojunction, the Schottky junction, metal-insulator-semiconductors structures, or semiconductors. For example, the semiconductor of silicon, or the like, absorbs the light to generate photo-carriers such as electrons and holes, and the photo-carriers drift outside due to an internal electric field of the semiconductor. The photovoltaic devices for converting light to electric energy have been commonly used as solar cells to supply power for consumer-oriented products. The present invention utilizes the photovoltaic device, such as the structure of the solar cell, to enhance biological operations involving various charged biological species. Since the charges can be induced on the surface of the photovoltaic device by illuminating the device, the charged biological species contained in a solution disposed on the device can be attracted onto the device surface. Biological interactions between the biological species that have been immobilized on the device surface and the charged biological species can be accelerated. Therefore, the time of hybridization would be dramatically shortened.

The invention is based on semiconductors, such as silicon, that are fabricated by photolithography techniques with 1˜2 photomasks. 10,000 microlocations of 0.1 mm×0.1 mm can be integrated into a chip of 2 cm×2 cm. Mass production of chips can be achieved with large-size semiconductor wafers. The simple photolithography techniques used in the fabrication process reduce the cost of the chips. The chip can be positioned on a chamber where the solution containing the biological species is filled. Light sources such as lasers or LED's are utilized to accelerate the biological operations. The light sources can also be integrated as a microarray to supply light to the microlocations of the chips. Microlens may be used to focus the light onto the microlocations to enhance the induced charge density. The stringency can be achieved by applying a current on the chip. This helps to remove the non-specific species from the chip.

2. Photovoltaic Matrix (Chip):

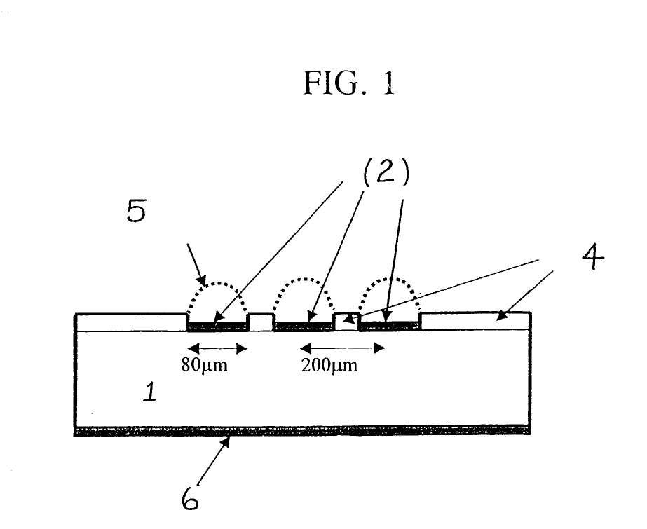

1) The structure of the chip: Substrate, Metal Film, Dielectric Film, As depicted in FIG. 1, a n-type silicon chip 1 is coated with a 50 Å-thick Ti layer and a 100 Å-thick Au layer 2 to form a Schottky contact in the structure of a metal-semiconductor solar cell. The metal thin film can be patterned by photolithography techniques to form an array of microlocations 2. 3 um-thick SiO2 film 4 is deposited on the chip and patterned by photolithography techniques to define and isolate the microlocations 2. Biological species 5 may be deposited at given microlocations 2. The backside of the chip 1 is coated with a 2000 Å-thick Al layer 6 that is used in conjunction with a gold electrode (not shown in FIG. 1) to apply a voltage to provide the desired stringency.

2) The Making of the Photovoltaic Device:

A titanium thin film and a gold thin film are deposited on the n-type silicon wafer sequentially. A spin coater deposits photoresist deposited on the wafer. A mask-aligner and a photomask are used to define the pattern of the microlocations on the photoresist. After the development of the photoresist and a hard baking step, a wet etching step transfers the pattern of the photomask onto the gold thin film. After this process, the microlocations contain a titanium/gold thin film and photoresist. A SiO₂ thin film is deposited on the wafer. The SiO₂ thin film is patterned by a lift-off technique. The SiO₂ on the photoresist that is localized on the microlocations is removed by acetone. After cutting the wafer into chips, the photovoltaic devices can be realized.

3) The Working Mechanism of the Photovoltaic Device

The deposition of gold on the n-type semiconductor bends the band structure of the silicon near the metal-semiconductor. This bending of the band structure induces an electric field. After light is directed onto the microlocation, a charge is induced in the semiconductor. The positive charges are pushed toward the semiconductor surface due to the electric field near the metal-semiconductor interface. These charges are concentrated on the metal thin film of the microlocations. The negative charges are moved toward the backside of the chip where it may be connected to a ground. The surface of the chip contains only the positive charges to enhance the biological operations.

3. Description of the Reaction Chamber, Light Source, Microlens

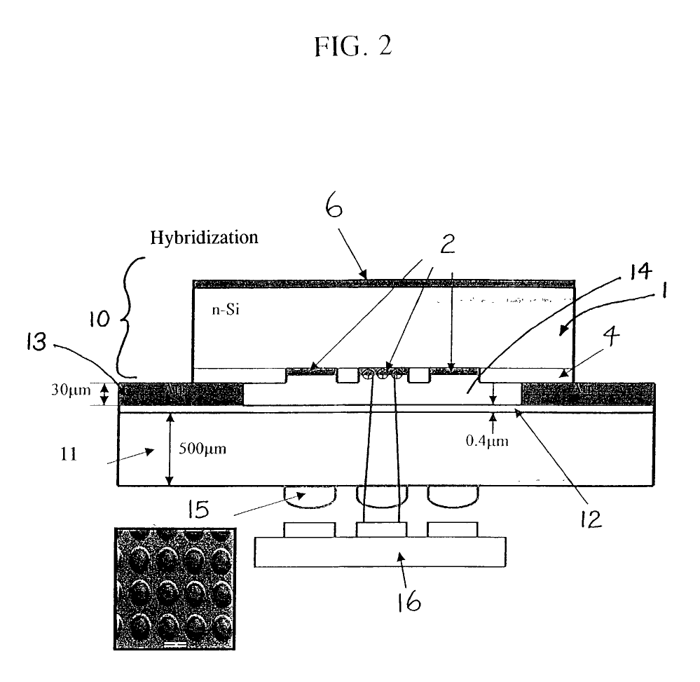

FIG. 2 shows a cross-section of the photovoltaic device 10 placed upside down from that displayed in FIG. 1, and on top of a transparent plate of sapphire 11 that is coated with a thin film of indium tin oxide 12 and a side border of gold having a thickness of 30 μm, 13. The photovoltaic device 10 and the plate 11 form a chamber 14, which is able to receive solutions containing biological materials. Microlens 15 at the bottom of the transparent plate 11 is able to focus light from a light source 16 onto the microlocations 2 of the photovoltaic device 10. A voltage may be applied to the gold electrodes 13 bordering the chamber 14 and the gold thin film at the microlocations 2 to provide stringency so as to remove species in solution such as unbound analytes and reactants.

The light source 16 may be a lamp, laser or LED. A transparent plate 11 such as glass, quartz or sapphire is placed between the light source and the microlocation. A transparent conductive thin film 12 such as Indium Tin Oxide (ITO) coats the surface of the transparent plate 11 facing the microlocation 2. 30 μm-thick Au 13 is deposited on selected areas on the ITO film 12 to form a chamber 14.

1) The device can be operated upside down if the chamber 14 is covered to isolate a solution in the chamber. Because the surface tension of the solution can avoid the lick-out of the solution in the chamber, the solution will be confined in the chamber.

2) The microlens 15 are optional and may or may not be used in the device of the present invention. The function of the microlens 15 is to focus the light from the light source 16, such as a laser or LED, onto the microlocations 2. If the light source 16 can create enough charges without focusing the light, the microlens 15 can be omitted. If we find the charges on the microlocation 2 is not enough or if we want to enhance the efficiency of the device, microlens 15 can help to achieve the desired effect.

4. Biological Operation of Stringency: Process and Mechanism

A light source 16 is not used in the operation of stringency. One embodiment of the present invention provides stringency by applying a negative charged field on the chip 1. An alternative way is using stringent washing buffers such as SSC and SDS to stringently discriminate the mismatched hybridization.

5. Hybridization: Process and Mechanism

Nucleic acid hybridization analysis generally involves the detection of a very small number of specific target nucleic acids (DNA or RNA) with an excess of probe DNA, and a relatively large amount of complex non-target nucleic acids. The actual hybridization reaction represents one of the most important and central steps in the whole process. The hybridization step involves placing the prepared DNA sample in contact with a specific reporter probe, at a set of optimal conditions for hybridization to occur to the target DNA sequence. Hybridization may be performed in any one of a number of formats. For example, multiple sample nucleic acid hybridization analysis has been conducted on a variety of filter and solid support formats (See G. A. Beltz et al., in Methods in Enzymology, Vol. 100, Part B, R. Wu, L. Grossman, K. Moldave, and Eds. Academic Press, New York, Chapter 19, pp. 266-308, 1985).

Immobilize Biological Species

Biological species such as oligonucleotides, cDNA, and proteins are immobilized on the gold surface through the use of an Easyspot coating layer (disclosed in U.S. Provisional Patent Application No. 60/314,936). In brief, the gold layer is coated with MCH, a molecule consisting of mercaptol and hydroxyl group, and then coated with EasySpot solutions. The biological species containing amine group will strongly bind to the epoxy groups on the EasySpot layer. The binding between amine and epoxy group is irreversible, making the biological species strongly immobilized on the gold surface.

Charged Biological Species

Many biological molecules are negatively charged. For example, phosphate groups on the backbone of DNA are negatively charged in most buffer systems. Proteins usually expose the hydrophilic groups outside which would be charged in a wide range of pH conditions. These charged molecules play essential roles in cell metabolism.

Transport to Microlocations

When the microlocations are exposed to a light source, the area will be temporally positively charged because of the electron holes generated by the photovoltaic effect. The positively charged surface of the microlocations will attract the negatively charged molecules, such as DNA, and increase the concentration of the biomolecules near the surface of the microlocations. Since the hybridization is a pseudo first order kinetics reaction, the increased concentration of reactants will apparently accelerate the rate of hybridizations.

The biological species can be deposited in the chamber. To focus the light on a given microlocation, an array of microlens can be fabricated on the transparent plate by photolithography technique. After the biological species is immobilized on the microlocations of the photovoltaic device, the photovoltaic device is placed upside down on a transparent plate. The light source is turned on to illuminate the microlocations, thereby inducing the charges to pull the charged biological species onto the surface of the microlocations. Biological operations such as hybridization can then be realized or accelerated.

6. Examples

1) Realization of Stringency

As depicted in FIG. 2, for stringency, the Al electrode 6 on the backside of the device and the Au electrode 13 on the chamber are connected with a current source. The electron accumulated on the microlocations 2 can remove the non-specific species on the microlocation.

2) Examples and data related to Hybridization,

Three genes, HPRT, G3PDH and TFR, were monitored for their efficiency and time for hybridization after illuminating the photovoltaic device with a laser source. The control that was used in the hybridization of these genes on a glass slide.

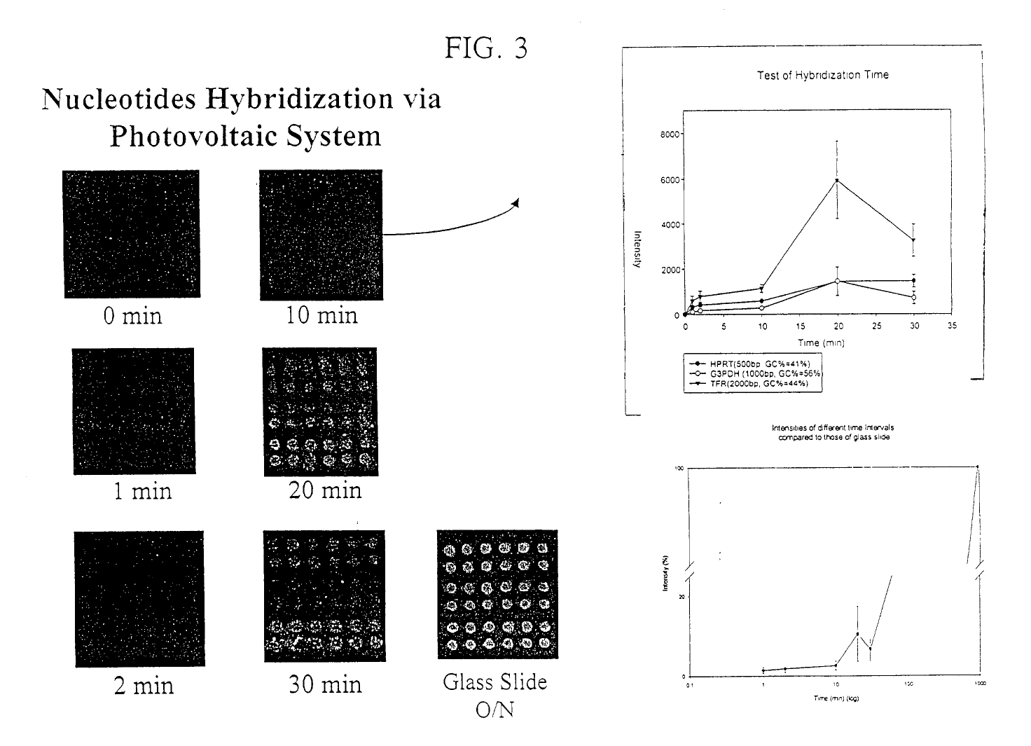

FIG. 3 shows a top view of the microlocations overtime as nucleotide hybridization proceeded at the microlocations while the microlocations were illuminated by a laser source. Fluorophore Cy3 was used as a signal indicator. The greater the fluorescence detected at the microlocation, the greater the degree of hybridization. Three genes (HTRP, G3PDH, and TFR) were hybridized at the microlocations and monitored for the rate of hybridization after illumination. After hybridization, the microlocations were washed with 2× SSC and 0.1% SDS followed by 0.2× SSC and 0.1% SDS. One-minute illumination resulted in more than 10% greater hybridization compared to the control, indicating that the photovoltaic device accelerated the interaction among the biomedical materials and increased the rate of hybridization. Illumination of greater than one minute results in even greater degrees of hybridization compared to the control. The results indicate that the illumination at the microlocations significantly increased the rate of hybridization.

The invention is not limited by the embodiments described above which are presented as examples only but can be modified in various ways within the scope of protection defined by the appended patent claims.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps, which perform substantially the same function in substantially the same way to achieve the same results, are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

We claim:

1. A photovoltaic device adapted for performing biological operations comprising:
a) a photovoltaic semiconductor chip, which comprises a photovoltaic semiconductor substrate, having a first side and a second side opposite the first side, and at least one microlocation formed by at least one metallic film disposed on the first side of the substrate to receive a solution comprising a biological sample;
b) a light source for providing photovoltaic energy to the microlocation and to subject the solution to an electrophoretic force.

2. The photovoltaic device of claim 1 wherein the microlocations on the photovoltaic semiconductor chip are arranged in an array and are isolated from each other by a dielectric material.

3. The photovoltaic device of claim 2 wherein the dielectric material is SiO₂.

4. The photovoltaic device of claim 1 wherein the second side of the substrate is coated with a metal film to effect stringency on the solution at the microlocation.

5. The photovoltaic device of claim 4 wherein the metal film is selected from the group consisting of gold aluminum, titanium, nickel, chrome, platinum, and alloys thereof.

6. The photovoltaic device of claim 1 further comprising a plate transparent to light and disposed on the microlocation to form a chamber.

7. The photovoltaic device of claim 6 wherein the plate is glass, quartz or sapphire.

8. The photovoltaic device of claim 6 wherein the plate is coated on the surface facing the microlocation with a transparent conductive thin film.

9. The photovoltaic device of claim 6 wherein the transparent conductive thin film is selected from the group consisting of indium tin oxide, indium oxide, tin oxide cadmium oxide, cadmium stannate and zinc stannate.

10. The photovoltaic devices of claim 1 wherein metallic layers are deposited on two opposite sides of the microlocation.

11. The photovoltaic device of claim 10 wherein the metallic layers comprises material selected from the group consisting of gold, aluminum, titanium, nickel, chrome, platinum, and alloys thereof.

12. The photovoltaic device of claim 1 further comprising a permeation layer disposed adjacent to the metallic film.

13. The photovoltaic device of claim 1 further comprising at least one lens between the light source and the microlocation to focus the light from the light source onto at least one microlocation.

14. The photovoltaic device of claim 13 where the light is focused on the side of the microlocation that receives the solution comprising the biological sample.

15. The photovoltaic device of claim 13 where the light is focused on the side of the microlocation opposite of the side of the microlocation that receives the solution comprising the biological sample.

16. A method of facilitating a biological operation comprising the steps of:
immobilizing a first biological species on the surface of a microlocation of a photovoltaic device of claim 1;
placing a solution comprising a second charged biological species into the microlocation; and
exposing the microlocation to light to create an electrophoretic force to move the charged second biological species toward the immobilized first biological species.

17. The method of claim 16, wherein the biological operation is selected from the group consisting of nucleic acid hybridization and antibody/antigen reaction.

18. The method of claim 16 further including the step of:
applying a voltage to the photovoltaic device to subject the solution to a second electrophoretic force to effect stringency on the solution at the microlocation.
